(19) United States
(12) Patent Application Publication
(10) Pub. No.: US 2004/0267244 A1
(43) Pub. Date: Dec. 30, 2004

(54) Medical instrument handle

(76) Inventors: Leonard Erickson, Lewisville, NC (US); Jack Calloway, Belew Creek, NC (US)

Correspondence Address: BRINKS HOFER GILSON & LIONE, P.O. BOX 10395, CHICAGO, IL, 60610, US

(21) Appl. No.: 10/846,122

(22) Filed: May 14, 2004

Related U.S. Application Data
(60) Provisional application No. 60/471,438, filed on May 16, 2003.

(51) Int. Cl. A61B 17/00

(52) U.S. Cl. 606/1

(57) ABSTRACT

A medical device is described, including a sheath and a handle with a slidable portion optionally having three finger rings for gripping the handle. The sheath has a protrusion adapted to mate with a recess in the distal end of the handle. A cap may optionally be attached to the distal end of the handle to prevent the sheath from inadvertently becoming dislodged in a lateral direction with respect to the handle. Using the handle, a user is able to operate a surgical instrument.

### BACKGROUND

1. Technical Field

This disclosure relates to a handle, and particularly to a handle used to operate a medical instrument.

2. Background Information

A variety of conventional handles, such as three-ring handles having a handle stem and a portion that slides relative to the handle stem, are used to operate catheter devices during various surgical procedures. These types of devices have a catheter sheath with a control wire extended there through. The distal end of the control wire is attached to a surgical instrument, for example a snare, and the proximal end is attached to a sliding portion of the handle, which is manipulated by a user to operate the surgical instrument. The catheter sheath is typically attached to the handle stem by first inserting the catheter sheath through a threaded cap, which is configured to engage a flared end of the catheter sheath. The cap is subsequently threaded onto the distal end of the handle stem. As a result, the flared end of the catheter sheath is compressed between the inside of the cap and the distal tip of the handle stem. That is, the inside of the cap and the distal tip of the handle stem create a retaining force that in part prevents the catheter sheath from pulling apart from the handle stem. To prevent the cap from inadvertently loosening and releasing the catheter sheath from the handle stem during use, glue is sometimes applied to permanently affix the cap to the distal end of the handle stem.

The above-described arrangement for securing the catheter sheath to the handle stem has a number of drawbacks. First, in order to secure the flared end of the catheter sheath to the handle, the cap is typically screwed onto the handle stem. This process is time-consuming and cumbersome, and ultimately increases the cost of manufacture for the handle. Second, since the flared end of the catheter sheath is held by the compressive force created between the cap and the handle stem, unintentionally loosing of the cap during a medical procedure may allow the catheter sheath to pull away from the handle, thus exposing a proximal portion of the control wire. Moreover, if the catheter sheath pulls away from the handle stem, the surgical instrument located at the distal end of the control wire may become obstructed or rendered inoperable. Third, a gap may form between the cap and the handle stem. This gap can become contaminated with bodily fluids or other materials. This gap also has an unappealing aesthetic quality.

### BRIEF SUMMARY

Accordingly, it is an object of the present invention to provide a medical device having features that resolve or improve upon one or more of the above-described drawbacks.

According to a first aspect of the present invention, the foregoing object is obtained by providing a medical device having a handle that has a recess adapted to grip the end of a sheath.

A medical device in accordance with yet another aspect of the present invention comprises an anchor, for example, a flange or flare, on the end of the sheath, wherein the handle has a recess adapted to grip the anchor on the end of the sheath.

A medical device in accordance with a further aspect of the present invention comprises a retaining member adapted to fit over the end of the handle, the handle having an interior portion that is adapted to grip the end of a sheath. The retaining member restricts the lateral movement of the sheath so as to retain the end of the sheath within the interior portion of the handle.

Still other objects and features of the present invention will become readily apparent to those skilled in this art from the following detailed description in conjunction with the accompanying drawings.

### BRIEF DESCRIPTION OF THE DRAWINGS

FIG. 1 is a perspective view of one embodiment of the present invention;

FIG. 2 is a partially exploded view of one embodiment of the present invention; and

FIG. 3 is a fully exploded view of one embodiment of the present invention.

### DETAILED DESCRIPTION OF THE DRAWINGS AND THE PRESENTLY PREFERRED EMBODIMENTS

The invention is described with reference to the drawings in which like elements are referred to by like numerals. The relationship and functioning of the various elements of this invention are better understood by the following detailed description. However, the embodiments of this invention as described below are by way of example only, and the invention is not limited to the embodiments illustrated in the drawings. It should also be understood that the drawings are not to scale and in certain instances details have been omitted, which are not necessary for an understanding of the present invention, such as conventional details of fabrication and assembly.

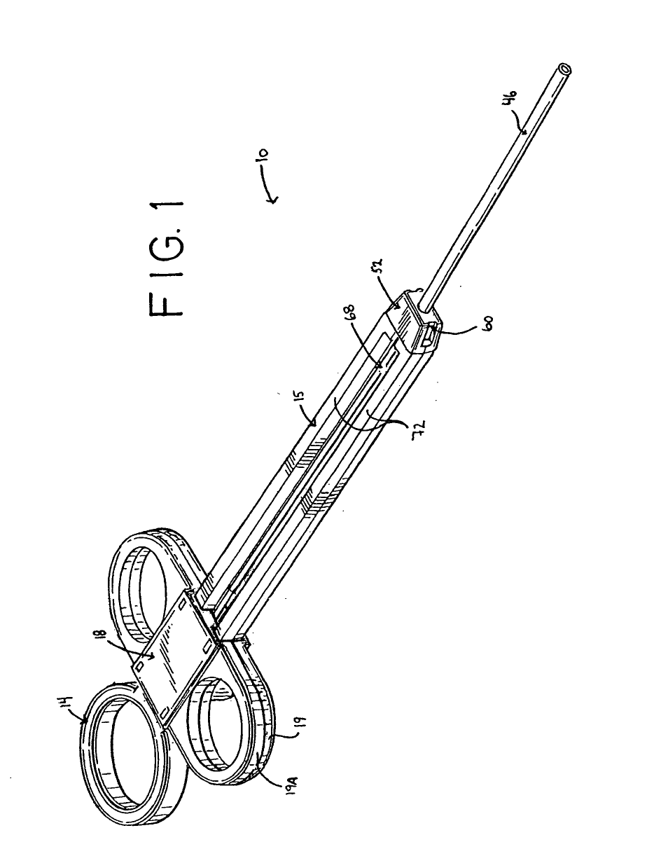

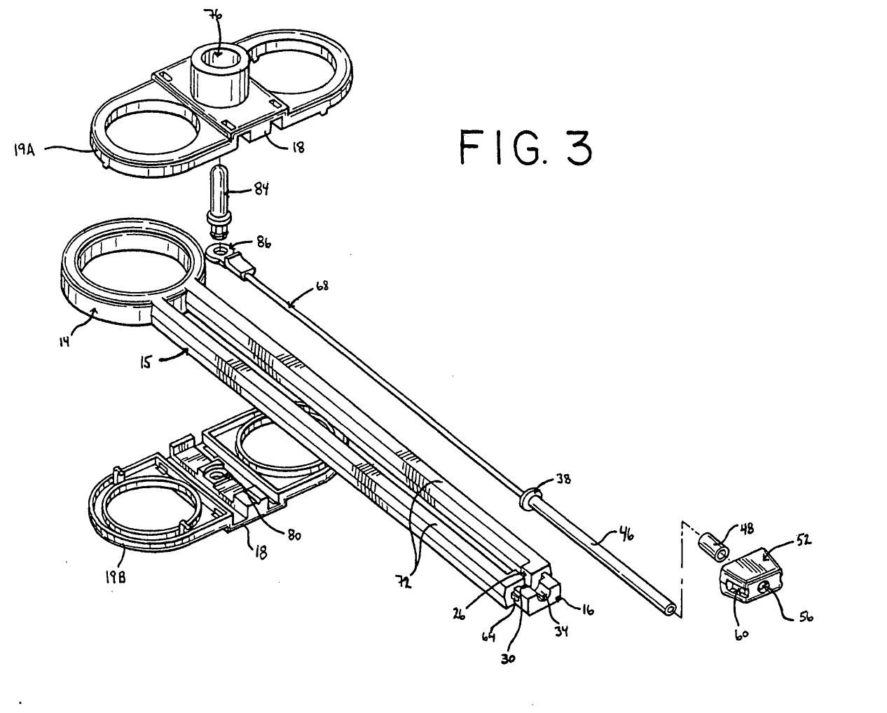

Referring to the drawings, FIGS. 1-3 illustrate a first embodiment of the present invention, and particularly, medical device 10. As best seen in FIG. 3, the medical device 10 generally comprises handle stem 15 having two slide rails 72, a thumb ring 14, sliding portion 18, and slot 30. The medical device 10 further comprises catheter sheath 46 having an expanded proximal end such as flange 38, control wire 68, and cap 52. Generally, flange 38 mates with slot 30 to secure catheter sheath 46 to distal end 16 of handle stem 15. Cap 52 fits over catheter sheath 46 and is secured to distal end 16 of handle stem 15.

Referring to FIG. 1, handle stem 15 is formed of a rigid material, such as a rigid plastic. Handle stem 15 has a proximal end, a central portion, and a distal end. The proximal end of handle stem 15 includes thumb ring 14, which allows a user to grip handle stem 15. Other conventional handle grips that facilitate gripping a handle stem may also be used, for example a handle having a pistol grip could be utilized. The central portion of the handle stem includes two parallel, spaced-apart slide rails 72. Slide rails 72 provide a surface for connecting sliding portion 18 to handle stem 15, and for allowing sliding portion 18 to slide axially along handle stem 15.

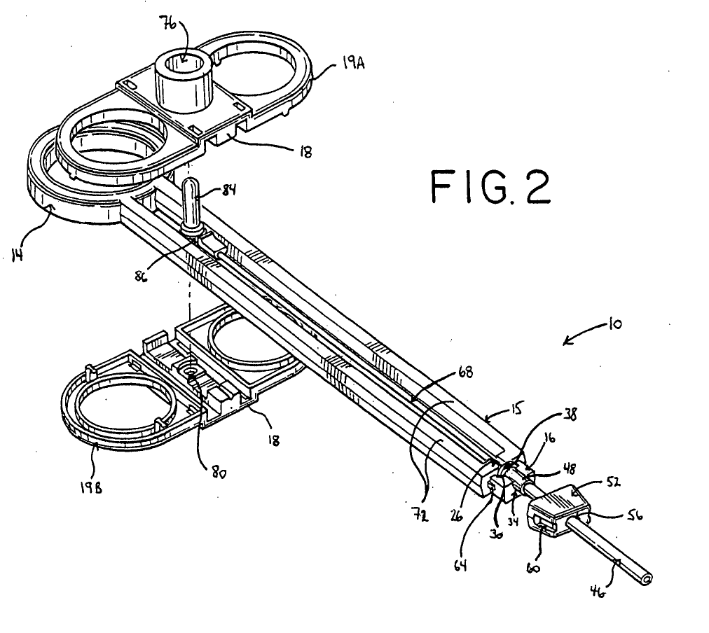

As best seen in FIG. 2, sliding portion 18 comprises an upper piece 19A and a corresponding lower piece 19B. As illustrated in FIG. 1, upper piece 19A and lower piece 19B may be snapped together over slide rails 72, thus connecting sliding portion 18 to handle stem 15. Sliding portion 18 includes a conventional pair of finger rings 19 configured to be gripped by a user's index and middle fingers. This configuration allows a user to slide sliding portion 18 axially relative to handle stem 15. Sliding portion 18 also includes port 76 for connecting a conventional cauterizing energy source (not shown) to control wire 68 through conductor stem 84. Sliding portion 18 further includes a conductor stem recess 80 for securing conductor stem 84 to sliding portion 18. Additionally, sliding portion 18 includes a notch 82 for securing anchor portion 86, which is located on the proximal end of control wire 68. When combined with handle stem 15, sliding portion 18 thus allows a user to move control wire 68 axially relative to sheath 46 so as to manipulate a surgical instrument, such as a snare wire loop, and to selectively energize the surgical instrument to cauterize or ablate a desired area.

The distal end of the handle stem includes a recess configured to engage and mate with the proximal end of the catheter sheath, and in particular, an anchor, expanded portion, or protrusion at the proximal end of the catheter sheath. It will become apparent to one of skill in this art that a multiplicity of recesses of different shapes and sizes may be utilized to engage and mate with the protrusion or expanded portion at the proximal end of the catheter sheath. For example, as illustrated in FIG. 3 of the illustrated embodiment of the present invention, the recess is slot 30. Slot 30 is configured to laterally receive and, preferably, to closely mate with flange 38 of catheter sheath 46. More specifically, the proximal and distal surfaces of flange 38 abut the proximal and distal surfaces of slot 30. As a result, when flange 38 is mated with slot 30 catheter sheath 46 is axially secured to handle stem 15. That is, any axial force created by the catheter sheath is transferred directly to the handle stem. Notably, it has been discovered that this configuration increases the pull-out force necessary to separate the catheter sheath from the handle stem.

In addition, the distal end of the handle stem includes a nesting surface for the body of the catheter sheath. In the embodiment illustrated in FIGS. 2-3, the nesting surface is sheath nest 34. Sheath nest 34 allows flange 38 to more fully mate with slot 30. Optionally, annular collar 48 may also be provided to ensure a snug fit between flange 38 and slot 30. However, sheath nest 34 may be configured to provide a snug fit for catheter sheath 46 without the use of annular collar 48. Distal end 16 of handle stem 15 further includes two catch surfaces 64, which are used to secure cap 52 to handle stem 15. Catch surfaces 64 are provided on opposing sides of distal end 16 of handle stem 15 and are configured to allow cap 52 to “snap-on” to distal end 16 of handle stem 15. Alternatively, distal end 16 and cap 52 may be screwed together by a conventional threaded connection. Distal end 16 also includes a control wire groove 26.

In the illustrated embodiment of the present invention, catheter sheath 46 is formed of a flexible material, such as polytetraflouroethylene (PTFE). Catheter sheath 46 has a passageway for receiving control wire 68. As illustrated in FIGS. 2-3, the proximal end of catheter sheath 46 includes flange 38. As noted above, different protrusions suitable for mating the proximal end of the catheter sheath with the recess provided at the distal end of the handle stem may also be used, such as a flared tip. Similarly, the flange 38 could have a non-circular cross-section such as, for example, a rectangular cross-section.

In the illustrated embodiment of the present invention, a retaining device such as, for example, a cap, a collar, or a sleeve, is used to prevent the proximal end of the catheter sheath from inadvertently dislodging laterally from the recess at the distal end of the handle stem. Cap 52 includes a concave surface that urges a portion of catheter sheath 46 against sheath nest 34. As a result of the concave surface, cap 52 prevents catheter sheath 46 from separating laterally from distal end 16 of handle stem 15. In other words, cap 52 prevents flange 38 from coming out of slot 30. In the embodiment illustrated in FIG. 3, cap 52 also includes a pair of opposing catch grooves 60 on opposing sides of cap 52. Catch grooves 60 correspond with catch surfaces 64, and allow cap 52 to “snap-on” to distal end 16. As a result, the proximal edges of cap 52 closely abut handle stem 15. That is, the proximal edge of the cap directly abuts the handle stem. Catheter sheath 46 is threaded through hole 56 in cap 52. In an alternative embodiment, a collar, sleeve or other

With reference to FIGS. 1-3, assembly of the medical device 10 involves threading control wire 68 through catheter sheath 46. The distal end of catheter sheath 46 is then threaded through hole 56. Flange 38 is subsequently mated with slot 30 and cap 52 is moved proximally over distal end 16 until catch grooves 60 “snap-on” to distal end 16. The control wire 68 is then secured to sliding portion 18 by inserting conductor stem 84 through loop 86 and securing conductor stem 84 to control wire fastener 80. Upper and lower pieces 19A, 19B, respectively, are then secured together to form sliding portion 18, such that conductor stem 84 is aligned with and passes through port 76.

It is therefore intended that the foregoing detailed description be regarded as illustrative rather than limiting, and that it be understood that it is the following claims, including all equivalents, that are intended to define the spirit and scope of this invention.

1. A medical device, comprising:
a sheath having a proximal end; and
a handle comprising an operative portion and an engaging portion, wherein the engaging portion defines a recess adapted to grip the proximal end of the sheath.

2. The medical device of claim 1, further comprising a control wire operably connected to the operative portion, wherein the engaging portion comprises a handle stem, and the operative portion is slidable in an axial direction relative to the handle stem.

3. The medical device of claim 2, further comprising a retaining member operatively connected to the engaging portion, wherein the retaining member restricts lateral movement of the sheath.

4. The medical device of claim 3, further comprising a snap-fit structure, wherein the retaining member is connected to the engaging portion by the snap-fit structure.

5. The medical device of claim 3, wherein the retaining member is screwed over the engaging portion.

6. The medical device of claim 2, further comprising a surgical instrument operably connected to the control wire, the surgical instrument being operable by the handle.

7. The medical device of claim 1, wherein the recess is a slot.

8. The medical device of claim 1, wherein the proximal end of the sheath comprises an expanded portion that corresponds with the recess.

9. The medical device of claim 8, wherein the expanded portion comprises a flared portion.

10. The medical device of claim 8, wherein the expanded portion comprises a flange.

11. A medical device, comprising:
a sheath comprising an anchoring portion;
a handle comprising a sliding member and a handle stem defining a recess adapted to grip the anchoring portion, wherein the sliding member is slidable in a direction axial to the handle stem; and
a retaining member operatively connected to the handle stem, wherein the retaining member secures the anchoring portion to the recess.

12. The medical device of claim 1 1, wherein the retaining member is a cap.

13. The medical device of claim 11, wherein the retaining member is a collar.

14. The medical device of claim 11, wherein the retaining member is a sleeve.

15. A method of assembling a medical device, said method comprising the steps of:
a) providing
a sheath having an expanded proximal end,
a handle comprising an operative portion and an engaging portion having a longitudinal axis, the engaging portion defining an interior volume adapted to grip the expanded proximal end, and
a retaining member adapted to secure the expanded proximal end within the interior volume defined by the engaging portion;
b) moving the expanded proximal end in a direction perpendicular to the longitudinal axis and into the interior volume defined by the engaging portion; and
c) securing the retaining member to the engaging portion.
